(19) United States
(12) Patent Application Publication
(10) Pub. No.: US 2025/0283110 A1
(43) Pub. Date: Sep. 11, 2025

(54) METHODS AND COMPOSITIONS FOR TRANSDUCTION OF NK CELLS

(71) Applicant: ST. JUDE CHILDREN'S RESEARCH HOSPITAL, INC., Memphis, TN (US)

(72) Inventors: Peter CHOCKLEY, Memphis, TN (US); Stephen GOTTSCHALK, Memphis, TN (US)

(21) Appl. No.: 18/564,987

(22) PCT Filed: May 27, 2022
(86) PCT No.: PCT/US2022/031269

§ 371 Date: Nov. 28, 2023

Related U.S. Application Data
(60) Provisional application No. 63/194,901, filed on May 28, 2021.

(51) Int. Cl. C12N 15/86; C12N 13/00; C12N 15/87

(52) U.S. Cl. CPC C12N 15/86; C12N 13/00; C12N 15/87; C12N 2740/15043

(57) ABSTRACT

Disclosed are methods for transducing cells, e.g., NK cells, comprising: contacting the cells with a transduction composition, e.g., a virus particle, and a TBK1/IKKε inhibitor, e.g., the TBK1/IKKε inhibitor can comprise a substituted N-(3-((2-((3-(aminomethyl)-phenyl)amino)-5-methylpyrimidin-4-yl)amino)propyl)acetamide analogue; thereby transducing the cells. The virus particle can comprises a lentiviral vector. The present disclosure also relates to kits comprising: a TBK1/IKKε inhibitor; and instructions for transducing NK cells; wherein the TBK1/IKKε inhibitor comprises a substituted N-(3-((2-((3-(aminomethyl)-phenyl)amino)-5-methylpyrimidin-4-yl)amino)propyl)acetamide analogue. This abstract is intended as a scanning tool for purposes of searching in the particular art and is not intended to be limiting of the present disclosure.

### CROSS-REFERENCE TO RELATED APPLICATIONS

This application is the 35 U.S.C. § 371 national stage application of PCT Application No. PCT/US2022/031269, filed May 27, 2022, where the PCT claims priority to, and the benefit of, U.S. provisional application No. 63/194,901, both of which are herein incorporated by reference in their entireties.

### STATEMENT REGARDING FEDERALLY SPONSORED RESEARCH

This invention was made with government support under award CA021765 awarded by the National Institutes of Health. The government has certain rights in the invention.

### BACKGROUND

Immunotherapy with genetically modified natural killer (NK) cells has garnered significant interest due to the recent encouraging clinical results of NK cells genetically modified to express CD19-specific chimeric antigen receptors (CARs) and interleukin (IL) 15 for patients with non-Hodgkin lymphoma (Refs. 1-2). In this clinical study and other preclinical studies, NK cells were genetically modified with GALV- or RD114-pseudotyped retroviral vectors (Ref. 2).

In addition to recombinant retroviruses, lentiviruses (LVs) are attractive vectors to genetically modify immune cells for adoptive immunotherapy. However, in contrast to T cells, the transduction efficiency of current Vesicular Stomatitis Virus G (VSV-G) pseudotyped LVs for NK cells has been disappointing (Ref. 3) Strategies have been described to improve the transduction efficiency of NK cells by VSV-G pseudotyped LVs (VSV-G LVs), including using protamine sulfate or upregulating the LDL receptor, the receptor required for LVs insertion (Refs. 4-5). In addition, a preclinical study demonstrated that inhibiting the antiviral pathway signaling through TBK1/IKKα/β/ε with a non-specific PDKI inhibitor, BX-795, resulted in increased transduction of NK cells (Ref. 6). More recently, several studies have highlighted that baboon retroviral envelope glycoprotein (BaEV) pseudotyped LVs allowed for efficient transduction of NK cells (Refs. 3, 7). Unfortunately, high titer viral preparations of BaEV-LVs are difficult to produce with standard protocols (Ref. 8) and no clinical study has been reported being conducted with BaEV-LV transduced cells. In general, conventional methods that are currently available to genetically modifying NK cells with VSV-G LVs have been variable and unreliable.

Despite advances in research directed to use of VSV-G pseudotyped lentiviral vectors (LVs) to NK cells, there is still a scarcity of methods that allow reliable, effective, and efficient transduction of NK cells by these vectors. These needs and other needs are satisfied by the present disclosure.

### SUMMARY

In accordance with the purpose(s) of the invention, as embodied and broadly described herein, the invention, in one aspect, relates to methods and compounds useful for generation of genetically modified NK cells using VSV-G LVs. In various aspects, the disclosed methods comprise inhibition of the IKK-related protein kinases, TBK1/IKKε, which are signaling molecules of the endosomal TLR4 pathway, which is activated by VSV-G. The disclosed methods enable the reliable transduction of NK cells by VSV-G LVs.

Disclosed are methods for method for transducing cells comprising: contacting cells with a transduction composition and a TBK1/IKKε inhibitor; and wherein the TBK1/IKKε inhibitor comprises a substituted N-(3-((2-((3-(aminomethyl)-phenyl)amino)-5-methylpyrimidin-4-yl)amino)propyl)acetamide analogue; thereby transducing the cells.

Also disclosed are methods for method for transducing NK cells comprising: contacting NK cells with a transduction composition and a TBK1/IKKε inhibitor; and wherein the TBK1/IKKε inhibitor comprises a substituted N-(3-((2-((3-(aminomethyl)-phenyl)amino)-5-methylpyrimidin-4-yl)amino)propyl)acetamide analogue; thereby transducing the NK cells.

Also disclosed are methods for transducing cells comprising: contacting cells with a virus particle and a TBK1/IKKε inhibitor; wherein the virus particle comprises a lentiviral vector; and herein the TBK1/IKKε inhibitor comprises a substituted N-(3-((2-((3-(aminomethyl)-phenyl)amino)-5-methylpyrimidin-4-yl)amino)propyl)acetamide analogue; thereby transducing the cells.

Also disclosed are methods for transducing NK cells comprising: contacting NK cells with a virus particle and a TBK1/IKKε inhibitor; wherein the virus particle comprises a lentiviral vector; and herein the TBK1/IKKε inhibitor comprises a substituted N-(3-((2-((3-(aminomethyl)-phenyl)amino)-5-methylpyrimidin-4-yl)amino)propyl)acetamide analogue; thereby transducing the NK cells.

Also disclosed are kits comprising: a TBK1/IKKε inhibitor; and instructions for transducing cells; wherein the TBK1/IKKε inhibitor comprises a substituted N-(3-((2-((3-(aminomethyl)-phenyl)amino)-5-methylpyrimidin-4-yl)amino)propyl)acetamide analogue.

Also disclosed are kits comprising: a TBK1/IKKε inhibitor; and instructions for transducing NK cells; wherein the TBK1/IKKε inhibitor comprises a substituted N-(3-((2-((3-(aminomethyl)-phenyl)amino)-5-methylpyrimidin-4-yl)amino)propyl)acetamide analogue.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show representative data for viral pathway inhibition allowing VSV-G LV transduction of NK cells. FIG. 1A a simplified schematic of endosomal TLR4 viral sensing pathway in NK cells with drug targeting believed to occur as indicated. FIG. 1B shows representative flow cytometry histogram plots of YFP expression in NK cells at highest used drugs concentrations (untreated: NK cells transduced in the absence of TBK1/IKKε inhibitors). FIG. 1C shows representative % YFP expression curves (N=3 mean±sem) in response to increasing drug concentrations; the half maximal effective concentration (EC₅₀) for each compound is shown in parentheses.

FIGS. 2A-2C show representative data demonstrating that CD19-CAR NK cells recognize and kill CD19-positive target cells. FIG. 2A shows a schematic representation of CD19-CAR with domains indicated as labeled therein. FIG. 2B shows representative flow cytometry data plotted as a histogram plot CD19-CAR expression with indicated MOIs (untreated: NK cells transduced in the absence of MRT67307 at MOI of 30). FIG. 2C shows representative flow cytometric based cytotoxicity assay data of CD19-CAR NK cells targeting leukemia cell lines BV173 and BV173.CD19KO at indicated effector to target (E: T) ratios; N=4 donors, averaged technical triplicates per donor, mean±sem; unpaired two-tailed student's t-test was used to determine significance, **p<0.01.

FIGS. 3A-3F show representative data demonstrating that viral pathway inhibition allows insertions of two transgenes into NK cells. FIG. 3A shows a schematic of HER2-CAR.2A.iMC expression cassette with domains/features as labeled therein (“TM” indicates “transmembrane”). FIG. 3B shows representative flow cytometry data demonstrating HER2-CAR expression detected with anti-F (ab′) 2. FIG. 3C shows representative Western blot analysis of HER2-CAR NK cells demonstrating that expression of HER2-CAR.CD37 and endogenous CD34 (anti-CD32); and iMC (anti-HA tag) expression. GAPDH was used a loading control. FIG. 4D shows representative cytotoxicity data (4-hour MTS assay) at an E:T ratio of 2:1, 1:1, and 0.5:1 with NK cells that were transduced with HER2-CAR LVs in the absence (untreated) or presence of MRT67307 (HER2-CAR) as effectors and A549 cells as targets, N=4 donors, averaged technical replicates per donor, mean±sem. Unpaired two-tailed student's t-test was used to determine significance, *p<0.05. FIG. 3E shows representative data of the percentage of NK cells that produced 2, 3, 4, or 5+ effector molecules of a 32-analyte panel. Chemical Inducer of Dimerization: CID was used to initiate iMC signaling. FIG. 3F shows representative Polyfunctional Strength Index information detailing effector, stimulatory, regulatory, chemo attractive, and inflammatory molecules; N=2 donors. FIG. 3G shows representative aggregate data of NK cells transduced in the absence (−) (N=18) or presence (+) (BX-795 N=4, MRT67307 N=9) of TBK1/IKKε inhibitors; mean±sem; *p<0.0001; Mann Whitney U-test.

FIGS. 4A-4B show representative data relating to NK cell phenotype with and without treatment with a representative disclosed compound, MRT67307. Briefly, NK cells were treated with and without 2μMMRT67307 for 24 hours. Media was removed and replaced with fresh complete media and cells incubated for another 48 hours. Cells were immunophenotyped via flow cytometry. NK cells were identified as efluor455UV negative, CD3 negative, and CD56 positive. No parameter was significantly different by paired two-tailed Student's t-Test between treated and untreated, N=3, mean±sem. FIG. 4A shows data obtained for cells that were not treated with MRT67307. FIG. 4B shows data obtained for cells that were treated with MRT67307.

FIG. 5 shows representative data comparing NK cell transduction techniques. Briefly, quantified FACS data detailing YFP expression using various conventional methods for NK cell transduction with VSV-G LVs; N=2 for No Virus; LentiBOOST™ (Sirion Biotech, Martinsried, Germany); BX795; LentiBOOST™+BX795; N=5 for Spinfection (Spin) Polybrene (8 μg/mL, Sigma-Aldrich); and Spin Alone. Spinfection was carried out at 850 g for 90 minutes at 32° C. MOI of 10 was used for all conditions. Data are mean±sem for all groups.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Reference to “a” chemical compound refers to one or more molecules of the chemical compound rather than being limited to a single molecule of the chemical compound. Furthermore, the one or more molecules may or may not be identical, so long as they fall under the category of the chemical compound. Thus, for example, “a” chemical compound is interpreted to include one or more molecules of the chemical, where the molecules may or may not be identical (e.g., different isotopic ratios, enantiomers, and the like).

As used in the specification and the appended claims, the singular forms “a,” “an” and “the” include plural referents unless the context clearly dictates otherwise. Thus, for example, reference to “a compound,” “a TBK1/IKKε inhibitor,” or “a kinase,” includes, but is not limited to, two or more such compounds, TBK1/IKKε inhibitors, or kinases, and the like.

Reference to “a/an” chemical compound, protein, and antibody each refers to one or more molecules of the chemical compound, protein, and antibody rather than being limited to a single molecule of the chemical compound, protein, and antibody. Furthermore, the one or more molecules may or may not be identical, so long as they fall under the category of the chemical compound, protein, and antibody. Thus, for example, “a” protein is interpreted to include one or more molecules of the protein, where the protein molecules may or may not be identical (e.g., different post-translational modifications as may be found in a protein).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent. For example, a compound contacting a protein refers to the compound being in proximity to the protein by the compound interacting and binding to the protein via ionic, dipolar and/or van der Waals interactions. In some instances, contacting can comprise both physical and chemical interactions between the indicated components. It is to be understood that chemical interactions can comprise a combination of covalent and non-covalent interactions, including one or more of ionic, dipolar, van der Waals interactions, and the like.

As used herein, the terms “optional” or “optionally” means that the subsequently described event or circumstance can or cannot occur, and that the description includes instances where said event or circumstance occurs and instances where it does not.

As used herein, a “cell” means a biological cell, particularly a mammalian cell, that is inclusive of cells both in vitro, e.g., a cultured or isolated cell, and in vivo, e.g., a cell in matrix such as blood or a cell in a tissue.

As used herein, an “inhibitor” is understood to mean a molecule which can reduce or inhibit the biological effect of another molecule, in particular a protein, e.g., a TBK1/IKKε kinase.

As used herein, “TBK1/IKKε” or “TBK1/IKKε kinase” can be used interchangeabley and refer to one or both of, TBK1 (TANK [TRAF (tumour-necrosis-factor-receptor-associated factor)-associated nuclear factor KB activator]-binding kinase 1) and IKKε which are IKK-related protein kinases. In the foregoing, “IKK” indicates IκB (inhibitor of nuclear factor κB) kinase. The IKK-related protein kinases, TBK1/IKKε, are noncanonical members of the inhibitor of the nuclear factor κB (IκB) kinase family and characterized as serine/threonine kinases.

As used herein, “transduction” of a cell means introducing a transduction complex into a cell. Transduction (or stated as a verb, “transducing”) comprises various methods or techniques of introducing the transduction composition into the cell such as electroporation, transfection, or other means as known to the skilled artisan. It is understood, unless specifically stated otherwise, that transduction is construed broadly and is inclusive of both viral-mediated transfer of a nucleic acid and/or protein to a cell (often referred to as “transduction” or “viral-mediated transduction”) and non-virus mediated transfer of a nucleic acid and/or protein to a cell (often referred to as “transfection”). Viral mediated transfer can include using lentivirus-mediated transduction, adenovirus-mediated transduction, retrovirus-mediated transduction, adeno-associated virus-mediated transduction or herpesvirus-mediated transduction, as well as cellular transfection as known to the skilled artisan. The process of transducing a cell can result in generation of “genetically modified cells”.

As used herein, “transduction composition” refers to exogeneous nucleic acids and/or proteins, as well as particles, complexes, or compositions comprising such exogeneous nucleic acids or proteins such as genetic material, e.g., RNA or DNA, a protein, nucleoprotein complex, and/or a virus particle into the cell. A virus particle can incorporate one or more nucleic acids into a virus particle for subsequent transfer into the cell via infection of the cell by the virus particle or means of transduction as disclosed herein.

As used herein, a RNP complex refers to a complex comprising a ribonucleic acid and a protein, e.g, a complex comprising a modified guide RNA such as the sgRNA and a Cas9 polypeptide or active fragment thereof. The RNP may be made by combining Cas9 and the desired guide RNA in a buffered aqueous solution as described herein or in accordance with known methods.

As used herein, CRISPR refers to the Clustered Regularly Interspaced Short Palindromic Repeats type II system used by bacteria and archaea for adaptive defense. This system enables bacteria and archaea to detect and silence foreign nucleic acids, e.g., from viruses or plasmids, in a sequence-specific manner. In type II systems, guide RNA interacts with Cas9 and directs the nuclease activity of Cas9 to target DNA sequences complementary to those present in the guide RNA. Guide RNA base pairs with complementary sequences in target DNA. Cas9 nuclease activity then generates a double-stranded break in the target DNA.

As used herein, CRISPR/Cas9 refers to an RNP complex. CRISPR RNA (crRNA) includes a 20 base protospacer element that is complementary to a genomic DNA sequence as well as additional elements that are complementary to the transactivating RNA (tracrRNA). The tracrRNA hybridizes to the crRNA and binds to the Cas9 protein, to provide an active RNP complex. Thus, in nature, the CRISPR/Cas9 complex contains two RNA species.

As used herein, sgRNA refers to a single RNA species which combines the tracrRNA and the crRNA and is capable of directing Cas9-mediated cleavage of target DNA. An sgRNA thus contains the sequences necessary for Cas9 binding and nuclease activity and a target sequence complementary to a target DNA of interest (protospacer sequence). In general, in an sgRNA, the tracrRNA and the crRNA are connected by a linker loop sequence. sgRNAs are well-known in the art. While sgRNA is generally used throughout this disclosure, two-part guide RNAs containing a crRNA and a tracrRNA can also be employed.

As used herein, “culturing” refers to the propagation of cells on or in media of various kinds. “Co-culturing” refers to the propagation of two or more distinct types of cells on or in media of various kinds.

As used herein, “gene delivery” refers to delivery of any exogenous nucleic acid molecule to a cell. Gene delivery methods encompassed by the present invention include, but are not limited to, “transduction” methods (i.e. virus-mediated transfer of nucleic acid to a target cell), and “transfection” methods (i.e. non-virus-mediated transfer of nucleic acid to a target cell).

The terms “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of single or double-stranded polynucleotides, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “vector” or “recombinant DNA vector” may be a construct that includes a replication system and sequences that are capable of transcription and translation of a polypeptide-encoding sequence in a given host cell. If a vector is used then the choice of vector is dependent upon the method that will be used to transform host cells as is well known to those skilled in the art. Vectors can include, without limitation, plasmid vectors and recombinant AAV vectors, or any other vector known in that art suitable for delivering a gene encoding a meganuclease of the invention to a target cell. The skilled artisan is well aware of the genetic elements that must be present on the vector in order to successfully transform, select and propagate host cells comprising any of the isolated nucleotides or nucleic acid sequences of the invention.

As used herein, a “vector” can also refer to a viral vector. Viral vectors can include, without limitation, retroviral vectors, lentiviral vectors, adenoviral vectors, and adeno-associated viral vectors (AAV).

As used herein, a “polycistronic” mRNA refers to a single messenger RNA that comprises two or more coding sequences (i.e., cistrons) and encodes more than one protein. A polycistronic mRNA can comprise any element known in the art to allow for the translation of two or more genes from the same mRNA molecule including, but not limited to, an IRES element, a T2A element, a P2A element, an E2A element, and an F2A element.

As used herein, a “human T cell” or “T cell” refers to a T cell isolated from a human donor. Human T cells, and cells derived therefrom, include isolated T cells that have not been passaged in culture, T cells that have been passaged and maintained under cell culture conditions without immortalization, and T cells that have been immortalized and can be maintained under cell culture conditions indefinitely.

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration that resulted in the genetically-modified cell; (b) a cell of the same genotype as the genetically-modified cell but that has been transformed with a null construct (i.e., with a construct that has no known effect on the trait of interest); or, (c) a cell genetically identical to the genetically-modified cell but that is not exposed to conditions or stimuli or further genetic modifications that would induce expression of altered genotype or phenotype.

As used herein, Cas9 polypeptide refers to Cas9 proteins and variants thereof having nuclease activity, as well as fusion proteins containing such Cas9 proteins and variants thereof. The fused proteins may include those that modify the epigenome or control transcriptional activity. The variants may include deletions or additions, such as, e.g., addition of one, two, or more nuclear localization sequences (such as from SV40 and others known in the art), e.g., 1, 2, 3, 4, 5, 6, 7, 8, 9, 10 such sequences or a range between and including any two of the foregoing values. In some aspects, the Cas9 polypeptide is a Cas9 protein found in a type II CRISPR-associated system. Suitable Cas9 polypeptides that may be used in the present technology include, but are not limited to, Cas9 protein from Streptococcus pyogenes (Sp. Cas9), F. novicida, S. aureus, S. thermophiles, N. meningitidis, and variants thereof. In some aspects, the Cas9 polypeptide is a wild-type Cas9, a nickase, or comprises a nuclease inactivated (dCas9) protein. In some aspects, the Cas9 polypeptide is a fusion protein comprising dCas9. In some aspects, the fusion protein comprises a transcriptional activator (e.g., VP64), a transcriptional repressor (e.g., KRAB, SID) a nuclease domain (e.g., FokI), a recombinase domain (e.g., Hin, Gin, or Tn3), a deaminase (e.g., a cytidine deaminase or an adenosine deaminase) or an epigenetic modifier domain (e.g., TET1, p300). In some aspects, the Cas9 polypeptide includes variants with at least 85% sequence identity, at least 90% sequence identity, at least 95% sequence identity, or even 96%, 97%, 98%, or 99% sequence identity to the wild type Cas9. Accordingly, a wide variety of Cas9 polypeptides may be used in the present nanocapsules as encapsulation is not sequence dependent so long as the surface of the Cas9 polypeptide has sufficient positive and/or negatively charged residues to allow coating by the anionic and/or cationic acryloyl-based monomers of the present technology. Other suitable Cas9 polypeptides may be found in Karvelis, G. et al. “Harnessing the natural diversity and in vitro evolution of Cas9 to expand the genome editing toolbox,” Current Opinion in Microbiology 37:88-94 (2017); Komor, A. C. et al. “CRISPR-Based Technologies for the Manipulation of Eukaryotic Genomes,” Cell 168:20-36 (2017); and Murovec, J. et al. “New variants of CRISPR RNA-guided genome editing enzymes,” Plant Biotechnol. J. 15:917-26 (2017), each of which is incorporated by reference herein.

Unless otherwise specified, temperatures referred to herein are based on atmospheric pressure (i.e., one atmosphere).

### B. Abbreviations

Abbreviations used herein are as follows: “LV” indicates lentiviral vector; “TM” indicates transmembrane, e.g., a protein transmembrane segment; “RNP” indicates a ribonucleoprotein complex and “YFP” indicates yellow fluorescent protein.

### C. Methods for Transduction of Cells

In one aspect, the disclosure relates to methods for transduction of cells with a transduction composition comprising contacting cells with a TBK1/IKKε inhibitor and a transduction composition, e.g., a virus particle comprising a VSG-G LV.

In one aspect, the disclosure relates to methods for transduction of NK cells with VSG-G LV comprising contacting cells with a TBK1/IKKε inhibitor and a virus particle, e.g., a virus particle comprising a VSG-G LV. In some instances, the cells are NK cells. In a further aspect, the cells are innate cells, e.g., γδ T cells.

In a further aspect, disclosed are methods transducing cells comprising: contacting cells, e.g., NK cells, with a virus particle and a TBK1/IKKε inhibitor; wherein the virus particle comprises a lentiviral vector; and herein the TBK1/IKKε inhibitor comprises a substituted N-(3-((2-((3-(aminomethyl)-phenyl)amino)-5-methylpyrimidin-4-yl)amino)propyl)acetamide analogue; thereby transducing the cells.

In a further aspect, the TBK1/IKKε inhibitor has an EC₅₀ of about 5 μM or less for transduction of the cells. Transduction can be determined using a virus particle comprising a transgene that act as a reporter, e.g., a yellow fluorescent protein. In a further aspect, the TBK1/IKKε inhibitor has an EC₅₀ of about 4 μM or less for transduction of the cells. In a still further aspect, the TBK1/IKKε inhibitor has an EC₅₀ of about 3 μM or less for transduction of the cells. In a yet further aspect, the TBK1/IKKε inhibitor has an EC₅₀ of about 2.5 μM or less for transduction of the cells. In an even further aspect, the TBK1/IKKε inhibitor has an EC₅₀ of about 2 μM or less for transduction of the cells. In a still further aspect, the TBK1/IKKε inhibitor has an EC₅₀ of about 1.5 μM or less for transduction of the cells.

In a further aspect, the TBK1/IKKε inhibitor has an IC₅₀ of less than about 200 nm for TBK1 kinase activity when assessed in an in vitro assay system. In a still further aspect, the TBK1/IKKε inhibitor has an IC₅₀ of less than about 100 nm for TBK1 kinase activity when assessed in an in vitro assay system. In a yet further aspect, the TBK1/IKKε inhibitor has an IC₅₀ of less than about 50 nm for TBK1 kinase activity when assessed in an in vitro assay system. In an even further aspect, the TBK1/IKKε inhibitor has an IC₅₀ of less than about 25 nm for TBK1 kinase activity when assessed in an in vitro assay system. In a still further aspect, the TBK1/IKKε inhibitor has an IC₅₀ of less than about 15 nm for TBK1 kinase activity when assessed in an in vitro assay system. In an even further aspect, the TBK1/IKKε inhibitor has an IC₅₀ of less than about 10 nm for TBK1 kinase activity when assessed in an in vitro assay system. In a still further aspect, the TBK1/IKKε inhibitor has an IC₅₀ of less than about 5 nm for TBK1 kinase activity when assessed in an in vitro assay system. In a yet further aspect, the TBK1/IKKε inhibitor has an IC₅₀ of less than about 2.5 nm for TBK1 kinase activity when assessed in an in vitro assay system. In an even further aspect, the TBK1/IKKε inhibitor has an IC₅₀ of about 0.5 nm to about 5 nM for TBK1 kinase activity when assessed in an in vitro assay system.

In a further aspect, the TBK1/IKKε inhibitor essentially does not inhibit kinases such IKKα/β, JAK and p38 MAPK up to about 10 μM when kinase activity is assessed in an in vitro assay system.

In a further aspect, the TBK1/IKKε inhibitor is a substituted N-(3-((2-((3-(aminomethyl)phenyl)amino)-5-methylpyrimidin-4-yl)amino)propyl)acetamide analogue such as a compound having a structure given by the formula:

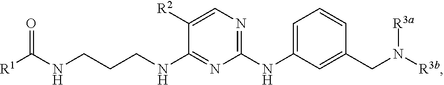

wherein R¹ is an alkyl, heteroalkyl, cycloalkyl, cycloheteroalkyl, aryl, heteroaryl, or substituted forms of any of the foregoing; wherein R² is an alkyl, heteroalkyl, cycloalkyl, cycloheteroalkyl, aryl, heteroaryl, or substituted forms of any of the foregoing; and wherein each of R^{3a} and R^{3b} are independently selected from hydrogen, alkyl, heteroalkyl, cycloalkyl, cycloheteroalkyl, aryl, heteroaryl, or substituted forms of any of the foregoing. The TBK1/IKKε inhibitor, i.e., a substituted N-(3-((2-((3-(aminomethyl)phenyl)amino)-5-methylpyrimidin-4-yl)amino)propyl)acetamide analogue, can be prepared by methods as known to the skilled artisan, e.g., as described by Mclver, E. G., et al. (2012) “Synthesis and structure-activity relationships of a novel series of pyrimidines as potent inhibitors of TBK1/IKKε kinases”, Bioorg. Med. Chem. Lett. 22, 7169-7173.

The disclosed methods allow reliable, effective, and efficient transduction of NK cells with a variety of VSG-G LV constructs, including constructs with relatively large inserts. Moreover, the disclosed methods utilize a single TBK1/IKKε inhibitor in order to effect the transduction, in contrast to some conventional methods that utilize both a TBK1/IKKε inhibitor and at least one additional agent, e.g., at least one inhibitor for at least one nucleic-acid-detecting toll-like receptor and/or a catioinic additive such as polybrene, protamine sulfate, and LentiBOOST™. The disclosed methods allow transduction of NK cells with a VSG-G LV construct without any additional agent such as a TLR inhibitor and/or a cationic additive.

In a further aspect, the TBK1/IKKε inhibitor is a substituted N-(3-((2-((3-(aminomethyl)phenyl)amino)-5-methylpyrimidin-4-yl)amino)propyl)acetamide analogue selected from one or more structure given by the following formulas:

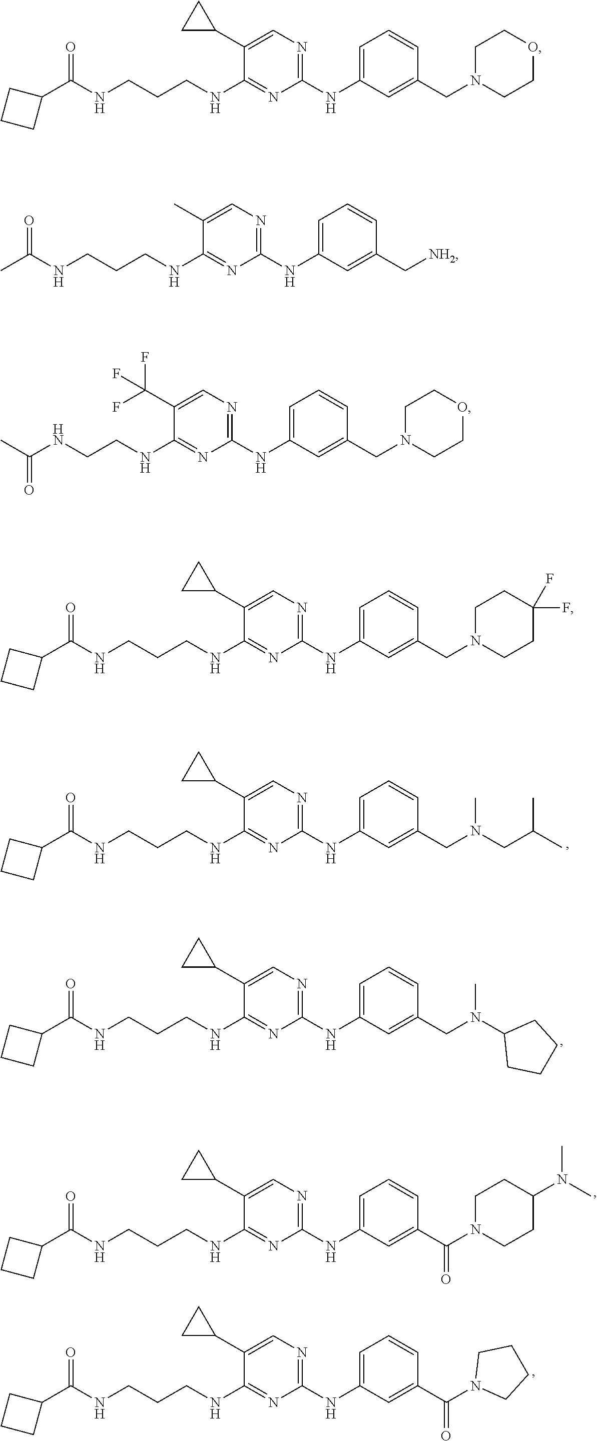

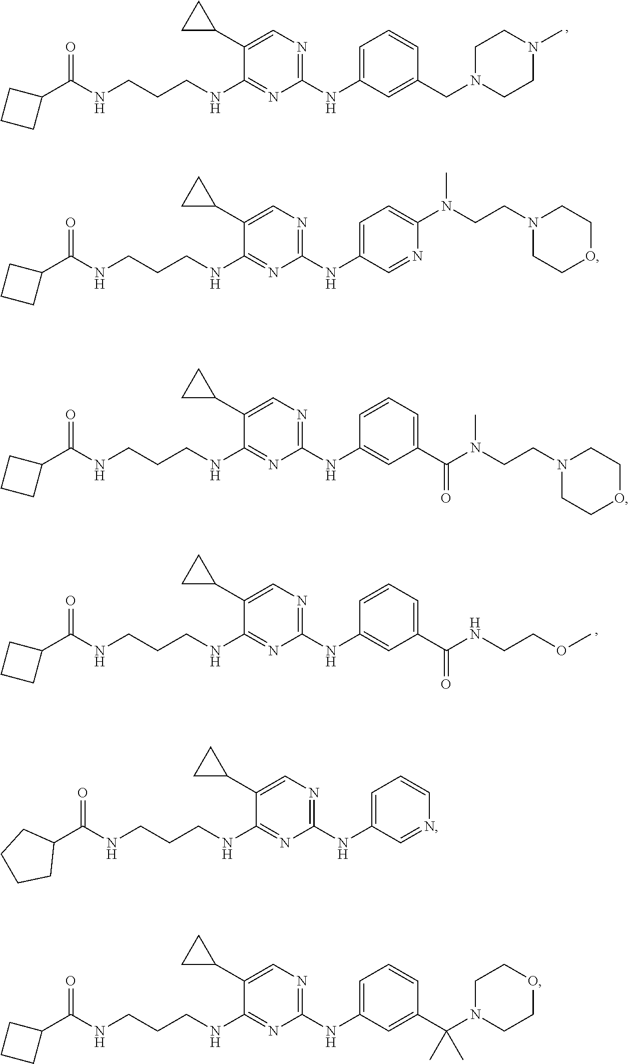 [SMILES: CN1CCN(Cc2cccc(Nc3ncc(C4CC4)c(NCCCNC(=O)C4CCC4)n3)c2)CC1 ; CN(CCN1CCOCC1)c1ccc(Nc2ncc(C3CC3)c(NCCCNC(=O)C3CCC3)n2)cn1 ; CN(CCN1CCOCC1)C(=O)c1cccc(Nc2ncc(C3CC3)c(NCCCNC(=O)C3CCC3)n2)c1 ; COCCNC(=O)c1cccc(Nc2ncc(C3CC3)c(NCCCNC(=O)C3CCC3)n2)c1 ; O=C(NCCCNc1nc(Nc2cccnc2)ncc1C1CC1)C1CCCC1 ; CC(C)(c1cccc(Nc2ncc(C3CC3)c(NCCCNC(=O)C3CCC3)n2)c1)N1CCOCC1]

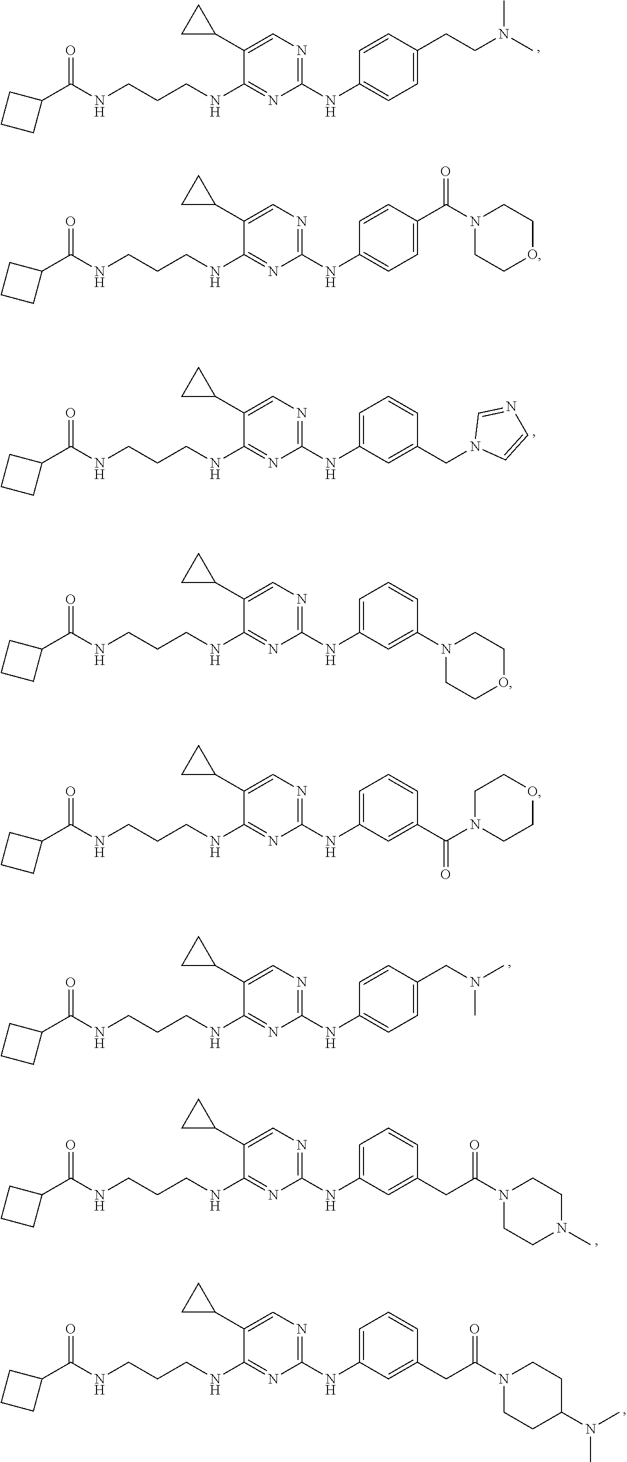

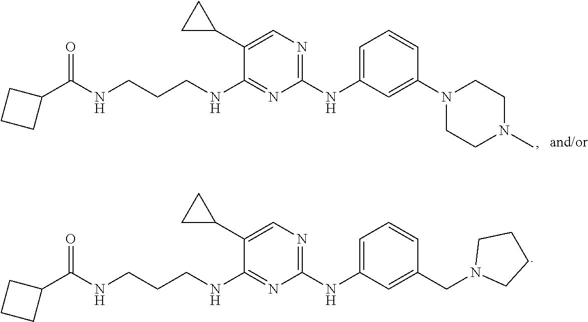 [SMILES: CN1CCN(c2cccc(Nc3ncc(C4CC4)c(NCCCNC(=O)C4CCC4)n3)c2)CC1 ; O=C(NCCCNc1nc(Nc2cccc(CN3CCCC3)c2)ncc1C1CC1)C1CCC1]

In a further aspect, the TBK1/IKKε inhibitor is a substituted N-(3-((2-((3-(aminomethyl)phenyl)amino)-5-methylpyrimidin-4-yl)amino)propyl)acetamide analogue associated with CAS number 1190378-57-4 and having a structure given by the formula:

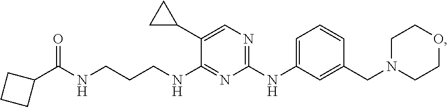 [SMILES: O=C(NCCCNc1nc(Nc2cccc(CN3CCOCC3)c2)ncc1C1CC1)C1CCC1]

which can also be referred to as MRT67307, MRT-67307, UNII-MEY37JZ4XR, MEY37JZ4XR, CHEMBL3605057, SCHEMBL13431845, N-[3-[[5-cyclopropyl-2-[3-(morpholin-4-ylmethyl) anilino]pyrimidin-4-yl]amino]propyl]cyclobutanecarboxamide, N-[3-[5-Cyclopropyl-2-[[3-(4-morpholinylmethyl)phenyl]amino]-4-pyrimidinyl]amino]propyl]cyclobutene-carboxamide, N-{3-[(5-Cyclopropyl-2-{[3-(Morpholin-4-Ylmethyl)phenyl]amino}pyrimidin-4-Yl)amino]propyl}cyclobutanecarboxamide, and other names as know to the skilled artisan. The foregoing compound will be referred to herein generally as MRT67307.

In a further aspect, the disclosed method does not utilize a cationic additive, such as, but not limited to, polybrene, protamine sulfate, and LentiBOOST™ (a proprietary transduction enhancer for lentiviral vectors; Sirion Biotech, Cambridge, Massachusetts). In a still further aspect, the disclosed method does not utilize an inhibitor of a toll-like receptor.

In a further aspect, the disclosed method of transducing a cell can utilize electroporation. In a still further aspect, In a further aspect, the disclosed method of transducing a call can utilize transfection.

A toll-like receptor (TLR for short) is understood to refer to proteins of the innate immune system. They belong to a group of receptors which are for recognising pathogenic structures and control corresponding gene activations. As a result, in particular the activation of the antigen-specific acquired immune system is initiated and modulated. By way of the toll-like receptors, the innate immune system can distinguish between “self” and “non-self”. More precisely, the TLRs are transmembrane proteins having an extracellular leucine-rich repeat (LRR) domain and a cytoplasmic domain, which is homologous with that of the IL-IR family. The various TLRs react selectively to different molecular viral and bacterial components, and control a corresponding activation of genes via a signal transduction cascade. This initially takes place by way of what are known as adapter molecules, and subsequently by way of kinases, which ultimately activate transcription factors (for example NF-κB and the IRF families), by phosphorylating them, or corresponding intracellular inhibitors of these transcription factors. Finally, alongside a large number of specific genes which have an antimicrobial effect, cytokines are produced. Cytokines are also necessary stimulators for the acquired immune system and thus also form a link between the innate and the acquired immune system. There are at least 13 different TLRs are known (of which 10 in humans), of which in turn only three are currently classified as nucleic-acid-detecting in humans: TLR3 (long dsRNA), TLR7 (ssRNA/dsRNA for example of RNA viruses) and TLR9 (bacterial/viral DNA).

A modified guide RNA is a one-part or two-part RNA capable of directing Cas-9-mediated cleavage of target DNA. A modified sg RNA is a single RNA species capable of directing Cas9-mediated cleavage of target DNA. A modified sgRNA, for example, comprises sequences that provide Cas9 nuclease activity, a protospacer sequence complementary to a target DNA of interest, and an aptamer that binds a biotin-binding molecule. The inventors of the present application unexpectedly found that the linker loop that connects the tracrRNA and the crRNA in an sgRNA can be replaced with an aptamer that binds a biotin-binding molecule such as a streptavidin-binding aptamer. Unexpectedly, the modified sgRNAs can bind both Cas9 protein and streptavidin, and form active RNP complexes which induce error-prone DNA repair less frequently than standard CRISPR-Cas9 RNP complexes.

In an aspect, a modified guide RNA, comprises a crRNA comprising a single-stranded protospacer sequence and a first complementary strand of a binding region for the Cas9 polypeptide, and a tracrRNA comprising a second complementary strand of the binding region for the Cas9 polypeptide, wherein the crRNA or the tracrRNA comprises an aptamer that binds a biotin-binding molecule, wherein the crRNA and the tracrRNA hybridize through the first and second complementary strands of the binding region for the Cas9 polypeptide.

In some aspects, the term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). In some aspects, a Cas9 nuclease has an inactive (e.g., an inactivated) DNA cleavage domain, that is, the Cas9 is a nickase. Other aspects of Cas9, both DNA cleavage domains are inactivated. This is referred to as catalytically-inactive Cas9, dead Cas9, or dCas9. Functional Cas9 mutants are described, for example, in US20170081650 and US20170152508, incorporated herein by reference for its disclosure of Cas9 mutants.

In some aspects, “transduced” can utilize electroporation. The electroporation may be, for example, flow electroporation or static electroporation. Electroporation of a transduction composition into cells, such as NK cells, results in conveying the transduction composition, or at least a portion of the transduction composition, intracellularly within the cells.

The disclosed methods of the present disclosure can be used to deliver transduction compositions, including exogenous nucleic acids or virus particles to, and/or to genetically modify, any desired target cell type. In some aspects the target cells are differentiated cells, such as, but not limited to, for example, lymphocytes, T-cells, B-cells, NK cells, myeloid cells, endocrine cells, mesenchymal cell, or epidermal cells. In some aspects the target cells are stem cells or progenitor cells, including, but not limited to pluripotent stem cells—such as embryonic stem cells (ES cells) or induced pluripotent stem cells (iPS cells). In some aspects the stem cells are tissue- or organ-restricted stem or progenitor cells—i.e. stem or progenitor cells that are committed to a certain cell or tissue lineage. For example, in some preferred aspects the target cells are hematopoietic stem cells (HSCs) or hematopoietic stem or progenitor cells (HSPCs). In some such aspects the HSCs or HSPCs are CD34-positive (CD34+). In some aspects the HSCs or HSPCs are derived from bone-marrow, peripheral blood, umbilical cord blood, amniotic fluid, or other sources of stem cells.

The step of transducing the cells with one or more transduction compositions, e.g., exogenous nucleic acid molecules or other molecules such as virus particles can be carried out using any suitable method known in the art. In some aspects this step is performed by transfection, for example using a method such as liposome-mediated transfection, polybrene-mediated transfection, DEAE dextran-mediated transfection, electroporation, calcium phosphate precipitation, microinjection, or micro-particle bombardment. In other aspects this step is performed by transduction with a virus, for example using lentivirus-mediated transduction, adenovirus-mediated transduction, retrovirus-mediated transduction, adeno-associated virus-mediated transduction or herpesvirus-mediated transduction.

The nucleic acid molecule delivered to the target cells using the methods described herein can be any nucleic acid molecule that is desired. For example, the nucleic acid molecule may encode a therapeutically useful protein, or a marker protein, or any other desired protein—without limitation. Similarly, the nucleic acid molecule delivered may not encode a protein. For example, it may encode a fragment of a protein, such as “gene-corrected” fragment of a nucleotide sequence present in the genome of the target cell, for example for use in correcting a genetic defect in the target cell. In some such methods the gene-corrected sequence may be used to replace a mutant sequence in the genome of the target cell, for example using one or more gene-editing/gene-correction technologies, including, but not limited to meganuclease, zinc finger nuclease, TALEN, and/or CRISPR-Cas more gene-editing/gene-correction technologies.

The process of transducing a cell can result in generation of “genetically modified cells”. These genetically modified target cells can be used as desired. For example, in some aspects the genetically modified target cells may be administered to a subject in need thereof, such as a human or non-human subject. In some such aspects the subject may have a disease or disorder affecting the target cells, for example a genetic disease or disorder, and/or a disease or disorder that results in a deficiency of the target cell population. In some preferred aspects the genetically modified target cells are HSCs or HSPCs and are administered to a subject having a disease or disorder that affects cells of the hematopoietic system. For example, in some aspects such a subject may have a deficiency in hematopoiesis caused by a myeloablative treatment. In other aspects such a subject may have a hematologic disease, an infectious immunodeficiency, an infectious disease affecting T cells, a genetic immunodeficiency, severe combined immunodeficiency, a genetic disease affecting erythrocytes, and/or an anemia-such as sickle cell anemia, Fanconi's anemia, or thalassemia. In some such aspects the target cells may be allogeneic with respect to the subject. In other such aspects the target cells may be autologous with respect to the subject.

When delivered to a cell, an exogenous nucleic acid of the disclosure can be operably linked to any promoter suitable for expression of the encoded polypeptide in the cell, including those mammalian promoters and inducible promoters previously discussed. An exogenous nucleic acid of the disclosure can also be operably linked to a promoter, e.g., a synthetic promoter. Synthetic promoters can include, without limitation, the JeT promoter (WO 2002/012514). In some aspects, the promoter can be a viral promoter such as endogenous promoters from the viral vector (e.g. the LTR of a lentiviral vector) or the well-known cytomegalovirus- or SV40 virus-early promoters. In a further aspect, genes are operably linked to a promoter that drives gene expression preferentially in the target cell (e.g., a human NK cell).

Exogenous nucleic acids of the disclosure may be introduced into the cell by any of the means previously discussed. In a particular aspect, exogenous nucleic acids are introduced by way of a viral vector, such as a lentivirus, retrovirus, adenovirus, or preferably a recombinant AAV vector. Recombinant AAV vectors useful for introducing an exogenous nucleic acid can have any serotype that allows for transduction of the virus into the cell and insertion of the exogenous nucleic acid sequence into the cell genome. In particular aspects, the recombinant AAV vectors have a serotype of AAV2 or AAV6. The recombinant AAV vectors can also be self-complementary such that they do not require second-strand DNA synthesis in the host cell.

In another particular aspect, an exogenous nucleic acid can be introduced into a cell using a single-stranded DNA template. The single-stranded DNA can comprise the exogenous nucleic acid and, in preferred aspects, can comprise 5′ and 3′ homology arms to promote insertion of the nucleic acid sequence into a nuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

Exogenous nucleic acids of the disclosure may be transduced into the cell by any of the means previously discussed. In a particular aspect, exogenous nucleic acids are introduced by way of a viral vector, such as a lentivirus, retrovirus, adenovirus, or preferably a recombinant AAV vector. Recombinant AAV vectors useful for introducing an exogenous nucleic acid can have any serotype that allows for transduction of the virus into the cell and insertion of the exogenous nucleic acid sequence into the cell genome. In particular aspects, the recombinant AAV vectors have a serotype of AAV2 or AAV6. The recombinant AAV vectors can also be self-complementary such that they do not require second-strand DNA synthesis in the host cell.

In a further aspect, the transduction is selected from electroporation, viral-mediated transduction, and a combination thereof.

### D. Methods of Treatment

In a further aspect, the present disclosure relates to methods of treating a subject, wherein the subject is administered an NK cell transduced by a disclosed method. In a further aspect, the subject is a patient having been diagnosed with a disorder of uncontrolled cellular proliferation, e.g., a cancer.

### E. Kits

In a further aspect, the present disclosure relates to kits comprising: a TBK1/IKKε inhibitor; and instructions for transducing NK cells; wherein the TBK1/IKKε inhibitor comprises a substituted N-(3-((2-((3-(aminomethyl)-phenyl)amino)-5-methylpyrimidin-4-yl)amino)propyl)acetamide analogue. In a further aspect, the kit further comprises a virus particle comprising a lentiviral vector. In a still further aspect, the kit further comprises NK cells. In a yet further aspect, the kit further comprises a lentiviral vector and NK cells. In various aspects, the instructions comprise instructions to a disclosed method.

### F. References

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
Ref 1. Liu, E. et al. Cord blood NK cells engineered to express IL-15 and a CD19-targeted CAR show long-term persistence and potent antitumor activity. Leukemia 32, 520-531, doi: 10.1038/leu.2017.226 (2018).
Ref 2. Liu, E. et al. Use of CAR-Transduced Natural Killer Cells in CD19-Positive Lymphoid Tumors. N Engl J Med 382, 545-553, doi: 10.1056/NEJMoa1910607 (2020).
Ref 3. Bari, R. et al. A Distinct Subset of Highly Proliferative and Lentiviral Vector (LV)-Transducible NK Cells Define a Readily Engineered Subset for Adoptive Cellular Therapy. Front Immunol 10, 2001, doi: 10.3389/fimmu.2019.02001 (2019).
Ref 4. Amirache, F. et al. Mystery solved: VSV-G-LVs do not allow efficient gene transfer into unstimulated T cells, B cells, and HSCs because they lack the LDL receptor. Blood 123, 1422-1424, doi: 10.1182/blood-2013-11-540641 (2014).
Ref 5. De Sanctis, J. B., Blanca, I., Radzioch, D. & Bianco, N. E. Expression and function of low-density lipoprotein receptors in CD3-CD16+CD56+ cells: effect of interleukin 2. Cell Immunol 167, 18-29, doi: 10.1006/cimm.1996.0003 (1996).
Ref 6. Sutlu, T. et al. Inhibition of intracellular antiviral defense mechanisms augments lentiviral transduction of human natural killer cells: implications for gene therapy. Hum Gene Ther 23, 1090-1100, doi: 10.1089/hum.2012.080 (2012).
Ref 7. Colamartino, A. B. L. et al. Efficient and Robust NK-Cell Transduction With Baboon Envelope Pseudotyped Lentivector. Front Immunol 10, 2873, doi: 10.3389/fimmu.2019.02873 (2019).
Ref 8. Bauler, M. et al. Production of Lentiviral Vectors Using Suspension Cells Grown in Serum-free Media. Mol Ther Methods Clin Dev 17, 58-68, doi: 10.1016/j.omtm.2019.11.011 (2020).
Ref 9. Georgel, P. et al. Vesicular stomatitis virus glycoprotein G activates a specific antiviral Toll-like receptor 4-dependent pathway. Virology 362, 304-313, doi: 10.1016/j.virol.2006.12.032 (2007).
Ref 10. Olejnik, J., Hume, A. J. & Muhlberger, E. Toll-like receptor 4 in acute viral infection: Too much of a good thing. PLOS Pathog 14, e1007390, doi: 10.1371/journal.ppat. 1007390 (2018).
Ref 11. Souza-Fonseca-Guimaraes, F. et al. Toll-like receptors expression and interferon-γ production by NK cells in human sepsis. Crit Care 16, R206, doi: 10.1186/cc11838 (2012).
Ref 12. Adib-Conquy, M., Scott-Algara, D., Cavaillon, J.-M. & Souza-Fonseca-Guimaraes, F. TLR-mediated activation of NK cells and their role in bacterial/viral immune responses in mammals. Immunology & Cell Biology 92, 256-262, doi: https://doi.org/10.1038/icb.2013.99 (2014).
Ref 13. Amaya, M., Keck, F., Bailey, C. & Narayanan, A. The role of the IKK complex in viral infections. Pathog Dis 72, 32-44, doi: 10.1111/2049-632X.12210 (2014).
Ref 14. Kravchenko, V. V., Mathison, J. C., Schwamborn, K., Mercurio, F. & Ulevitch, R. J. IKKi/IKKepsilon plays a key role in integrating signals induced by pro-inflammatory stimuli. J Biol Chem 278, 26612-26619, doi: 10.1074/jbc.M303001200 (2003).
Ref 15. Imai, C., Iwamoto, S. & Campana, D. Genetic modification of primary natural killer cells overcomes inhibitory signals and induces specific killing of leukemic cells. Blood 106, 376-383, doi: 10.1182/blood-2004-12-4797 (2005).
Ref 16. Gundry, M. C. et al. Highly Efficient Genome Editing of Murine and Human Hematopoietic Progenitor Cells by CRISPR/Cas9. Cell Rep 17, 1453-1461, doi: 10.1016/j.celrep.2016.09.092 (2016).
Ref 17. Chan, W. K. et al. Chimeric antigen receptor-redirected CD45RA-negative T cells have potent antileukemia and pathogen memory response without graft-versus-host activity. Leukemia 29, 387-395, doi: 10.1038/leu.2014.174 (2015).
Ref 18. Mata, M. et al. Inducible Activation of MyD88 and CD40 in CAR T Cells Results in Controllable and Potent Antitumor Activity in Preclinical Solid Tumor Models. Cancer Discov 7, 1306-1319, doi: 10.1158/2159-8290.CD-17-0263 (2017).
Ref 19. Liu, D., Paczkowski, P., Mackay, S., Ng, C. & Zhou, J. Single-Cell Multiplexed Proteomics on the IsoLight Resolves Cellular Functional Heterogeneity to Reveal Clinical Responses of Cancer Patients to Immunotherapies. Methods Mol Biol 2055, 413-431, doi: 10.1007/978-1-4939-9773-2_19 (2020).
Ref 20. Clark, K. et al. Novel cross-talk within the IKK family controls innate immunity. Biochem J 434, 93-104, doi: 10.1042/BJ20101701 (2011).
Ref 21. Clark, K., Plater, L., Peggie, M. & Cohen, P. Use of the pharmacological inhibitor BX795 to study the regulation and physiological roles of TBK1 and IkappaB kinase epsilon: a distinct upstream kinase mediates Ser-172 phosphorylation and activation. J Biol Chem 284, 14136-14146, doi: 10.1074/jbc.M109.000414 (2009).
Ref 22. Reilly, S. M. et al. An inhibitor of the protein kinases TBK1 and IKK-varepsilon improves obesity-related metabolic dysfunctions in mice. Nat Med 19, 313-321, doi: 10.1038/nm.3082 (2013).
Ref 23. Velasquez, M. P. et al. CD28 and 41BB Costimulation Enhances the Effector Function of CD19-Specific Engager T Cells. Cancer Immunol Res 5, 860-870, doi: 10.1158/2326-6066.CIR-17-0171 (2017).
Ref 24. Ahmed, N. et al. Regression of experimental medulloblastoma following transfer of HER2-specific T cells. Cancer Res 67, 5957-5964, doi: 10.1158/0008-5472.CAN-06-4309 (2007).
Ref 25. Micucci, F. et al. High-efficient lentiviral vector-mediated gene transfer into primary human NK cells. Exp Hematol 34, 1344-1352, doi: 10.1016/j.exphem.2006.06.001 (2006).
Ref 26. Gong, Y. et al. Rosuvastatin Enhances VSV-G Lentiviral Transduction of NK Cells via Upregulation of the Low-Density Lipoprotein Receptor. Mol Ther Methods Clin Dev 17, 634-646, doi: 10.1016/j.omtm.2020.03.017 (2020).
Ref 27. Tran, J. & Kung, S. K. Lentiviral vectors mediate stable and efficient gene delivery into primary murine natural killer cells. Mol Ther 15, 1331-1339, doi: 10.1038/sj.mt.6300184 (2007).
Ref 28. Boissel, L. et al. Comparison of mRNA and lentiviral based transfection of natural killer cells with chimeric antigen receptors recognizing lymphoid antigens. Leuk Lymphoma 53, 958-965, doi: 10.3109/10428194.2011.634048 (2012).
Ref 29. Hauber, I. et al. Improving Lentiviral Transduction of CD34 (+) Hematopoietic Stem and Progenitor Cells. Hum Gene Ther Methods 29, 104-113, doi: 10.1089/hgtb.2017.085 (2018).
Ref 30. Simon, B. et al. Enhancing lentiviral transduction to generate melanoma-specific human T cells for cancer immunotherapy. J Immunol Methods 472, 55-64, doi: 10.1016/j.jim.2019.06.015 (2019).
Ref 31. Jamali, A. et al. Highly Efficient Generation of Transgenically Augmented CAR NK Cells Overexpressing CXCR4. Front Immunol 11, 2028, doi: 10.3389/fimmu.2020.02028 (2020).
Ref 32. Bell, J. Amlexanox for the treatment of recurrent aphthous ulcers. Clin Drug Investig 25, 555-566, doi: 10.2165/00044011-200525090-00001 (2005).
Ref 33. Kudchodkar, S. B. & Levine, B. Viruses and autophagy. Rev Med Virol 19, 359-378, doi: 10.1002/rmv.630 (2009).
Ref 34. Fisher, J. & Anderson, J. Engineering Approaches in Human Gamma Delta T Cells for Cancer Immunotherapy. Front Immunol 9, 1409, doi: 10.3389/fimmu.2018.01409 (2018).
Ref 35. Vantourout, P. & Hayday, A. Six-of-the-best: unique contributions of gammadelta T cells to immunology. Nat Rev Immunol 13, 88-100, doi: 10.1038/nri3384 (2013).

### G. Aspects

The following listing of exemplary aspects supports and is supported by the disclosure provided herein.

Aspect 1. A method for transducing cells comprising: contacting cells with a virus particle and a TBK1/IKKε inhibitor; wherein the virus particle comprises a lentiviral vector; and wherein the TBK1/IKKε inhibitor comprises a substituted N-(3-((2-((3-(aminomethyl)-phenyl)amino)-5-methylpyrimidin-4-yl)amino)propyl)acetamide analogue; thereby transducing the cells.

Aspect 2. The method of Aspect 1, wherein the virus particle is a VSV-G pseudotyped LV particle.

Aspect 3. The method of Aspect 1 or Aspect 2, wherein the contacting the cells with a virus particle is at a multiplicity of infection (MOI) of about 1 to about 100.

Aspect 4. The method of Aspect 3, wherein the MOI is about 1 to about 10.

Aspect 5. The method of Aspect 3, wherein the MOI is about 2 to about 7.

Aspect 6. The method of Aspect 3, wherein the MOI is about 1 to about 5.

Aspect 7. The method of any one of Aspect 1-Aspect 6, wherein the substituted N-(3-((2-((3-(aminomethyl)phenyl)amino)-5-methylpyrimidin-4-yl)amino)propyl)acetamide analogue has a structure given by the formula:

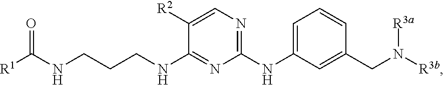

wherein R¹ is an alkyl, heteroalkyl, cycloalkyl, cycloheteroalkyl, aryl, heteroaryl, or substituted forms of any of the foregoing; wherein R² is an alkyl, heteroalkyl, cycloalkyl, cycloheteroalkyl, aryl, heteroaryl, or substituted forms of any of the foregoing; and wherein each of R^{3a} and R^{3b} are independently selected from hydrogen, alkyl, heteroalkyl, cycloalkyl, cycloheteroalkyl, aryl, heteroaryl, or substituted forms of any of the foregoing.

Aspect 8. The method of Aspect 7, wherein the substituted N-(3-((2-((3-(aminomethyl)-phenyl)amino)-5-methylpyrimidin-4-yl)amino)propyl)acetamide analogue has a structure given by the formula:

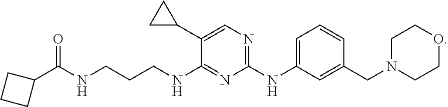 [SMILES: O=C(NCCCNc1nc(Nc2cccc(CN3CCOCC3)c2)ncc1C1CC1)C1CCC1]

Aspect 9. The method of any one of Aspects 1-Aspect 8, wherein about 10% to about 100% of the NK cells contacting the virus particle and the TBK1/IKKε inhibitor are transduced with the virus particle.

Aspect 10. The method of Aspect 9, wherein about 30% to about 100% of the cells contacting the virus particle and the TBK1/IKKε inhibitor are transduced with the virus particle.

Aspect 11. The method of Aspect 9, wherein at least about 30% of the cells contacting the virus particle and the TBK1/IKKε inhibitor are transduced with the virus particle.

Aspect 12. The method of Aspect 9, wherein at least about 40% of the cells contacting the virus particle and the TBK1/IKKε inhibitor are transduced with the virus particle.

Aspect 13. The method of Aspect 9, wherein at least about 50% of the cells contacting the virus particle and the TBK1/IKKε inhibitor are transduced with the virus particle.

Aspect 14. The method of any one of Aspect 1-Aspect 13, wherein the lentiviral vector comprises at least one transgene.

Aspect 15. The method of Aspect 14, wherein the transgene is from about 100 base pairs to about 5,000 base pairs.

Aspect 16. The method of any one of Aspect 1-Aspect 15, wherein the method does not utilize a toll-like receptor inhibitor.

Aspect 17. The method of any one of Aspect 1-Aspect 16, wherein the method does not utilize a cationic additive.

Aspect 18. The method of any one of Aspects 1-Aspect 17, wherein the cells comprise NK cells.

Aspect 19. The method of any one of Aspect 1-Aspect 18, wherein the cells comprise innate cells.

Aspect 20. The method of Aspect 19, wherein the innate cells are γδ T cells.

Aspect 21. The method of any one of Aspect 1-Aspect 52, wherein the transducing comprises electroporation.

Aspect 22. A kit comprising: a TBK1/IKKε inhibitor; and instructions for transducing cells; wherein the TBK1/IKKε inhibitor comprises a substituted N-(3-((2-((3-(aminomethyl)-phenyl)amino)-5-methylpyrimidin-4-yl)amino)propyl)acetamide analogue.

Aspect 23. The kit of Aspect 22, further comprising a virus particle comprising a lentiviral vector.

Aspect 24. The kit of Aspect 22 or Aspect 23, further comprising NK cells.

Aspect 25. The kit of any one of Aspect 22-Aspect 24, wherein the instructions comprise the method of any one of Aspects 1-Aspect 21.

Aspect 26. A method for transducing cells comprising: contacting cells with a transduction composition and a TBK1/IKKε inhibitor; and wherein the TBK1/IKKε inhibitor comprises a substituted N-(3-((2-((3-(aminomethyl)-phenyl)amino)-5-methylpyrimidin-4-yl)amino)propyl)acetamide analogue; thereby transducing the cells.

Aspect 27. The method of Aspect 26, wherein the transduction composition comprises a virus particle.

Aspect 28. The method of Aspect 27, wherein the virus particle comprises a lentiviral vector.

Aspect 29. The method of Aspect 28, wherein the lentiviral vector comprises at least one transgene.

Aspect 30. The method of Aspect 29, wherein the transgene is from about 100 base pairs to about 5,000 base pairs.

Aspect 31. The method of any one of Aspect 27-Aspect 30, wherein the virus particle is a VSV-G pseudotyped LV particle.

Aspect 32. The method of any one of Aspect 27-Aspect 31, wherein about 10% to about 100% of the cells contacting the virus particle and the TBK1/IKKε inhibitor are transduced with the virus particle.

Aspect 33. The method of Aspect 32, wherein about 30% to about 100% of the cells contacting the virus particle and the TBK1/IKKε inhibitor are transduced with the virus particle.

Aspect 34. The method of Aspect 32, wherein at least about 30% of the cells contacting the virus particle and the TBK1/IKKε inhibitor are transduced with the virus particle.

Aspect 35. The method of Aspect 32, wherein at least about 40% of the cells contacting the virus particle and the TBK1/IKKε inhibitor are transduced with the virus particle.

Aspect 36. The method of Aspect 32, wherein at least about 50% of the cells contacting the virus particle and the TBK1/IKKε inhibitor are transduced with the virus particle.

Aspect 37. The method of any one of Aspect 27-Aspect 36, wherein the contacting the cells with a virus particle is at a multiplicity of infection (MOI) of about 1 to about 100.

Aspect 38. The method of Aspect 37, wherein the MOI is about 1 to about 10.

Aspect 39. The method of Aspect 37, wherein the MOI is about 2 to about 7.

Aspect 40. The method of Aspect 37, wherein the MOI is about 1 to about 5.

Aspect 41. The method of any one of Aspect 26-Aspect 40, wherein the transduction composition comprises a ribonucleoprotein (RNP) complex.

Aspect 42. The method of Aspect 41, wherein the ribonucleoprotein (RNP) complex is a CRISPR ribonucleoprotein (RNP) complex.

Aspect 43. The method of any one of Aspect 26-Aspect 42, wherein the transduction composition comprises a protein.

Aspect 44. The method of Aspect 43, wherein the protein is an antibody.

Aspect 45. The method of any one of Aspect 26-Aspect 44, wherein the transduction composition comprises a plasmid, DNA fragment, oligonucleotide, RNA, or combinations thereof.

Aspect 46. The method of any one of Aspect 26-Aspect 45, wherein the substituted N-(3-((2-((3-(aminomethyl)phenyl)amino)-5-methylpyrimidin-4-yl)amino)propyl)acetamide analogue has a structure given by the formula:

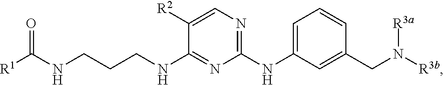

wherein R1 is an alkyl, heteroalkyl, cycloalkyl, cycloheteroalkyl, aryl, heteroaryl, or substituted forms of any of the foregoing; wherein R2 is an alkyl, heteroalkyl, cycloalkyl, cycloheteroalkyl, aryl, heteroaryl, or substituted forms of any of the foregoing; and wherein each of R3a and R3b are independently selected from hydrogen, alkyl, heteroalkyl, cycloalkyl, cycloheteroalkyl, aryl, heteroaryl, or substituted forms of any of the foregoing.

Aspect 47. The method of Aspect 46, wherein the substituted N-(3-((2-((3-(aminomethyl)-phenyl)amino)-5-methylpyrimidin-4-yl)amino)propyl)acetamide analogue has a structure given by the formula:

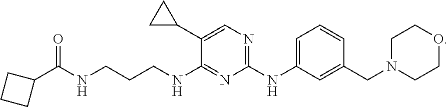 [SMILES: O=C(NCCCNc1nc(Nc2cccc(CN3CCOCC3)c2)ncc1C1CC1)C1CCC1]

Aspect 48. The method of any one of Aspect 26-Aspect 47, wherein the method does not utilize a toll-like receptor inhibitor.

Aspect 49. The method of any one of Aspect 26-Aspect 48, wherein the method does not utilize a cationic additive.

Aspect 50. The method of any one of Aspect 26-Aspect 49, wherein the cells comprise NK cells.

Aspect 51. The method of any one of Aspect 26-Aspect 50, wherein the cells comprise innate cells.

Aspect 52. The method of Aspect 51, wherein the innate cells comprise γδ T cells.

Aspect 53. The method of any one of Aspect 26-Aspect 52, wherein the transducing comprises electroporation.

Aspect 54. The method of any one of Aspect 26-Aspect 52, wherein the transducing comprises transfection.

Aspect 55. A kit comprising: a TBK1/IKKε inhibitor; and instructions for transducing cells; wherein the TBK1/IKKε inhibitor comprises a substituted N-(3-((2-((3-(aminomethyl)-phenyl)amino)-5-methylpyrimidin-4-yl)amino)propyl)acetamide analogue.

Aspect 56. The kit of Aspect 55, further comprising a virus particle comprising a lentiviral vector.

Aspect 57. The kit of Aspect 55 or Aspect 56, further comprising cells, wherein the cells are optionally TK cells.

Aspect 58. The kit of any one of Aspect 55-Aspect 57, wherein the instructions comprise the method of any one of Aspect 26-Aspect 53.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### H. Examples

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Methods.

a. Cell Lines.

BV173, and A549 were obtained and grown as per American Type Culture Collection (ATCC, Manassas, VA, USA) instructions. K562 with modified membrane bound interleukin (IL)-15 and 4-1BB ligand (Ref. 15), feeder cells, were a generous gift from Dr. Dario Campana (National University of Singapore) and grown in IMDM media with 10% fetal bovine serum (FBS; Hyclone Laboratories, Chicago, IL, USA). CD19 BV173 cell line were generated with CRISPR/Cas9 technology using a published method (Ref. 16).

b. Generation of Lentiviral Vectors.

The generation of the CD19-CAR.4-1BB.CD3ζ LV was previously described (Ref. 17); this LV is also used in our current clinical study to generate clinical grade CD19-CAR T cells (NCT03573700). The same LV backbone, except that the insulators were removed from the self-inactivating 3′ partially deleted viral long terminal repeats, was used to generate the LV encoding HER2-CAR.CD3ζ.2A.IMC. This expression cassette was subcloned from a previously published retroviral vector (Ref. 18) and is under control of the MND promoter (myeloproliferative sarcoma virus enhancer, negative control region deleted, dl587rev primer-binding site substituted). The cloned construct was verified by sequencing at the Hartwell Center at St. Jude Children's Research Hospital (St. Jude). Purified VSV-G pseudotyped LV particles were produced by the St. Jude Vector Core Laboratory by using transient transfection, followed by fast protein liquid chromatography purification) Ref. 8). The St. Jude Vector Core Laboratory also provided VSV-G pseudotyped LV particles encoding YFP.

### C. NK Cell Activation, Expansion, and Genetic Modification.

Human peripheral blood mononuclear cells (PBMCs) were obtained from whole blood of healthy donors under an IRB approved protocol at St. Jude, after informed consent was obtained in accordance with the Declaration of Helsinki. Cells were subjected to ACK Red Blood cell lysis and Ficcol Hypaque (Sigma-Aldrich, St. Louis, MO, USA) gradient separation. Cellular subtype analysis was performed with BD whole blood analysis kit on a BD Lyric flow cytometer (Becton-Dickinson, Franklin Lakes, NJ, USA). Cells were aliquoted and Freezing Media with 10% DMSO at 1×10⁷ cells per mL and stored in liquid nitrogen vapor phase until use. 150 Gray cesium-irradiated feeder cells were added to thawed PBMCs at a ratio of 5-10:1 feeder to NK cells as determined previously. Cells were grown in Stemcell Genix (20802-0500, Cellgenix, Portsmouth, MA, USA) growth media with 20% FBS and 10 Units/mL of IL-2, (Peprotech, Rocky Hill, NJ, USA). After 5-7 days cells were phenotyped and used for downstream experiments.

Genetically modified NK cells were generated as follows. 500,000 NK cells were seeded per well of a 24 well tissue culture plate in a volume of 1 mL of complete growth media. Inhibitors BX795, MRT67307, and Amlexanox (702675-74-9, 1190378-57-4, and 68302-57-8, Cayman Chemical, Ann Arbor, MI, USA) were added at indicated concentrations for 30 minutes and LVs were then added at indicated MOIs. NK cells were incubated for 24 hours and then media was removed and replaced with 2 mL of complete growth media. NK cells where then assessed for transgene expression 72 hours later.

### D. Western Blot.

Cell lysates were prepared with RIPA buffer with protease inhibitor cocktail. 20 μg of total protein were loaded in 12% pre-cast gels (Biorad Laboratories, Hercules, CA, USA) gels were transferred with the iBlot2 system with run template PO (Applied Biosystems, Foster City, CA, USA). Antibodies used were anti-CD3ζ (6B10.2, Santa Cruz Biotechnology, 1:1,000) with anti-Mouse HRP (NA931, Cytiva, Chicago, IL, USA; 1:10,000), Anti-GAPDH-HRP (GAPDH 71.1; catalog G9295, Sigma-Aldrich; 1:10,000), anti-HA (A190-108A, Bethyl Laboratories; 1:1000) with anti-Rabbit HRP (NA934V, Cytiva; 1:10,000) Were used for chemiluminescent detection digitally imaged on a Li-COR machine (Li-COR, Lincoln, NE, USA).

### E. Flow Cytometry.

250,000 NK cells were collected and washed twice in DPBS. Surface HER2-CAR detection was determined via immunolabeling with anti-F (ab′) 2-AF647 (109-606-006, Jackson Labs, Bar Harbor, ME, USA; 1:100), CD19-CAR was detected with PE conjugated recombinant human CD19 protein (3309HP, Creative Biomart, Shirley, NY, USA; 1:100), or YFP were utilized for detection on a BD FACS Lyric machine and analyzed with FlowJo v10 (BD). Immunophenotyping was performed on a BD Symphony flow cytometer. Antibodies used are delineated in below in Table 1.

TABLE 1
Antibodies utilized for NK immunophenotyping.

| SPECIFICITY | CLONE | FLUOROCHROME | COMPANY | CAT# |
|---|---|---|---|---|
| CD3 | OKT3 | Superbright 600 | Thermofisher | 63-0037-42 |
| CD56 | NCAM16.2 | PE-TX red | BD Biosciences | 564849 |
| CD62L | DREG-56 | BB515 | BD Biosciences | 464742 |
| NKG2D | ID11 | PerCPeFlour710 | Thermofisher | 46-5878-42 |
| CD57 | NK-1 | PE | BD Biosciences | 560844 |
| CD16 | REA423 | APC-H7 | BD Biosciences | 560715 |
| NKG2C | REA205 | PE Vio 770 | Miltenyi Biotech | 130-119-776 |
| Live/Dead | | eFlour455UV | Thermofisher | 65-0868-18 |

### F. Isolight Cytokine Detection.

Briefly, 500,000 NK cells were labeled with cell trace violet 1:1000 (ThermoFisher Scientific, Waltham, MA, USA) co-cultured at a 2:1 ratio with A549 targets for 4 hours in a 24 well plate. NK cells were removed and washed twice with PBS and resuspended in complete growth media without IL-2. These cells were loaded onto a single cell secretomic chip, IsoLight, that detects 32 distinct proteins (Ref. 19). Results were analyzed on IsoSpeak version 2.7.0.0 (IsoPlexis, Branford, CT, USA).

### G. Cytotoxicity Assays.

A549: 10,000 A549 cells as targets for HER2-CAR NK cells, cocultures were set at 2:1, 1:1, and 0.5:1 effector to target (E: T) ratios for 4 hours in a 96 well plate. Cytotoxicity was quantified by a chromogenic MTS assay measured on a plate reader (Tecan, Männedorf, Switzerland) detecting remaining viable adherent tumor cells. Virally transduced, but MRT67307 untreated NK cells were used as controls (untreated). BV173: 50,000 BV173 cancer cells as targets for CD19-CAR NK cells, BV173 cancer cells were labeled with CFSE (ThermoFisher, 1:500) and co-cultures were set at indicated ratios in 200 μL and after 4-hours of co-incubation, 3000 BD countbright beads were added to each well and assessed via flowcytometry for remaining CFSE positive BV173 cells. Samples were acquired until 100 beads were recorded. The percentage of remaining CFSE tumor cells compared to control ‘no NK cells’ wells was calculated to the according following equation:

$$100 - ( \frac{CFSE Experimental}{CFSE Control} ) \times 100 ) .$$

### 2. Results.

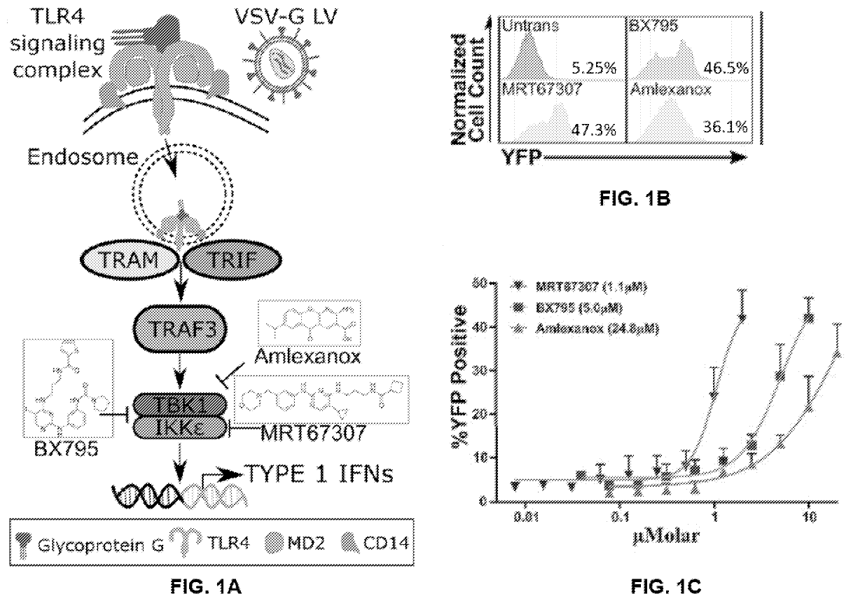

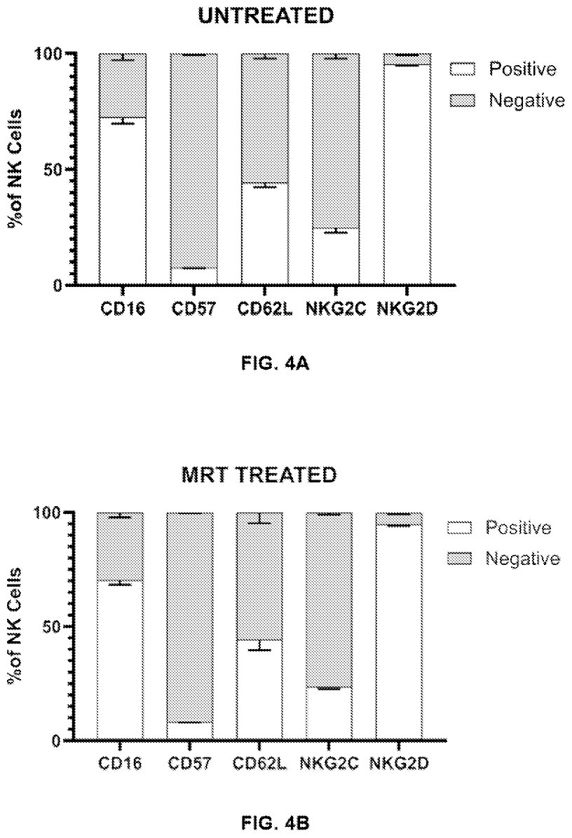

The feasibility of modifying NK cells with VSV-G LVs was explored for molecules that transiently block TBK1/IKKε, downstream of the endosomal TLR4 pathway, which is activated by VSV-G (Refs. 9-10). An overview of this pathway and drug compounds used in the disclosed study are depicted in FIG. 1A. NK cells were generated from PBMCs by employing standard expansion techniques using irradiated K562 cells, expressing membrane bound IL-15 and 4-1BB ligand, in the presence of exogenous IL-2. NK cells were then transduced with VSV-G LVs encoding a yellow fluorescent protein (YFP) at an MOI of 10 on days 5 to 7 post initial activation and selective expansion. Concurrent with viral particle addition, increasing concentrations of inhibitors were added. After 24 hours, inhibitors and LV containing media was removed and replaced with fresh NK cell growth media. The present disclosed study evaluated three compounds: MRT67307 (Ref. 20), BX-795 (Ref. 21), and Amlexanox (Ref. 22). All drugs block at least TBK1/IKKε (FIG. 1A) with MRT67307, a derivative of BX-795, being more specific for TBK1/IKKε than BX-795 (Ref. 20). Transduction efficacy was determined by FACS analysis 72 hours post transduction (FIG. 1B). In the presence of all three compounds, NK cells expressed YFP in a dose dependent manner with an EC₅₀ of 1.1 μM for MRT67307, 5 μM for BX-795, and 24.8 μM for Amlexanox (FIG. 1C). In view of these results, the compound with the lowest EC₅₀, MRT67307, at a 2 μM concentration was used in the subsequent studies detailed herein. It was determined that at this concentration (2 μM) MRT67307 treatment had no effect on NK cell phenotype (FIGS. 4A-4B).

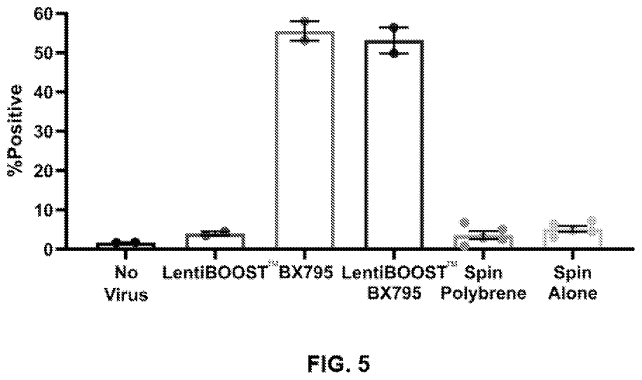

Cationic additives such as polybrene, protamine sulfate, and LentiBOOST™ have been utilized to enhance transduction efficiencies of VSV-G LVs for other cell types (Refs. 29-30). The next study assessed the effect of LentiBOOST™ in the presence of TBK1/IKKε inhibition, and it was determined that LentiBOOST™ did not further enhance transduction efficiencies (FIG. 5). Since LentiBOOST™ by itself did not enhance transduction efficiencies of NK cells, this result was further confirmed for a 2nd cationic additive (polybrene) (FIG. 5).

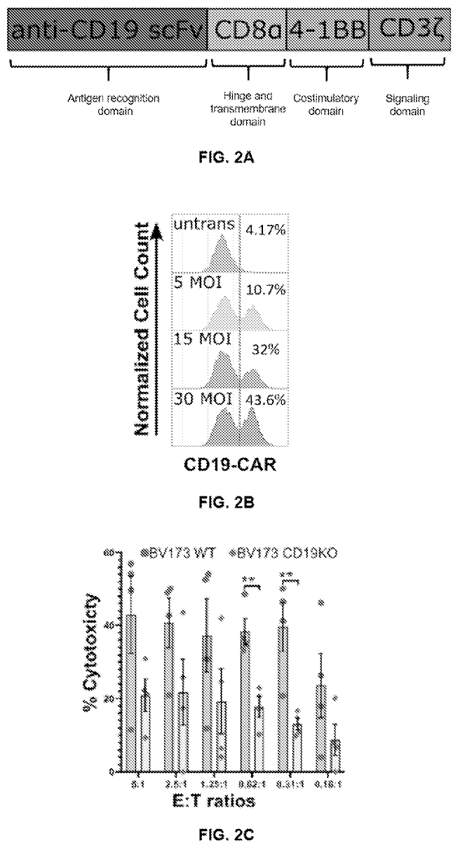

The studies disclosed herein also examined insertion of larger constructs into NK cells using our the disclosed methods. A CD19-CAR designed to target CD19+ malignancies with a 4-1BB.CD3ζ signaling domain (Ref. 23; see present FIG. 2A) was used in these studies. A range of MOIs were utilized and similar transduction efficiencies as determined by FACS analysis for CAR expression were observed (FIG. 2B). To validate CD19-CAR functionality, the cytolytic activity of CD19-CAR NK cells against the CD19+ leukemia cell line, BV173 (BV173.wt) was examined. Furthermore, to show target antigen specificity, BV173 in which CD19 was knocked out by CRISPR/Cas9 gene editing (BV173.CD19KO) was used. CD19-CAR NK cells were capable of targeting BV173 cells regardless of CD19 expression at high effector to target ratios during a 4-hour co-culture assay. However, expression of CD19-CAR in NK cells enabled them to kill CD19+BV173 cells, BV173.wt, at a very low effector to target ratio (0.62 and 0.31) in contrast to BV173.CD19KO cells (FIG. 2C).

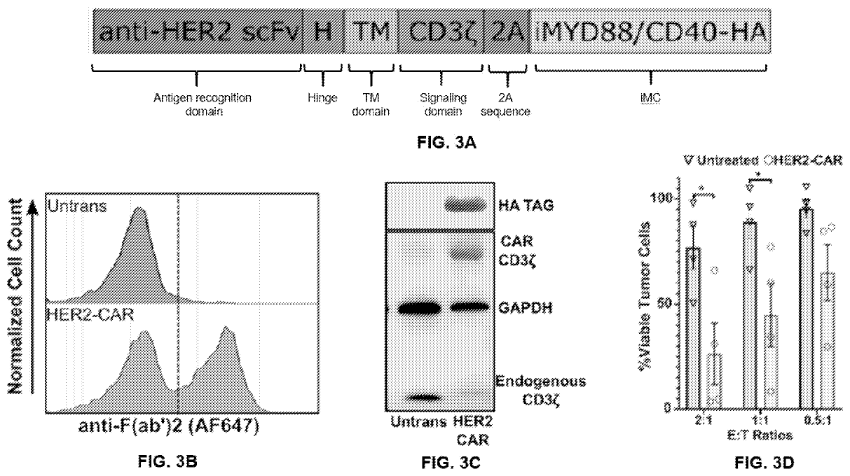

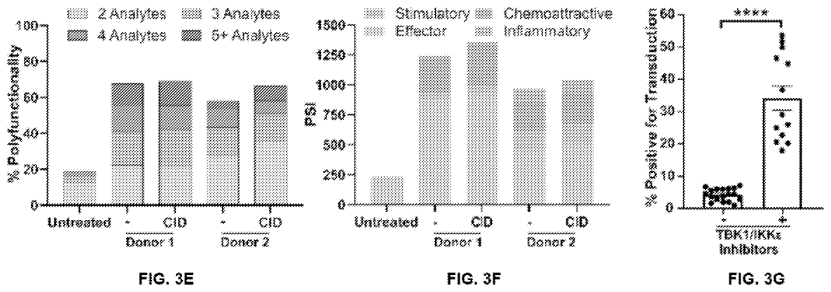

The next set of studies disclosed herein insert two genes, encoded by a single vector, into NK cells. The LV construct encoded a 1^{st} generation HER2-CAR²⁴, a 2A peptide, and an inducible MyD88/CD40 (IMC)¹⁸ costimulatory molecule (FIG. 3A). This entire vector insert is approximately 2.8 kilobases in length. The surface CAR expression was analzyed by flow cytometry and the expression of the full-length CAR and iMC validated via western blot. In this study, approximately 50% transduction of NK cells was achieved as judged by CAR cell surface expression (FIG. 3B); in addition, western blot demonstrated CAR and iMC expression (FIG. 3C).

Further, CAR functionality was assessed in a 4-hour cytotoxicity assay against the HER2+A549 (lung cancer adenocarcinoma) cell line. At an E:T ratio of 2:1 and 1:1, HER2-CAR NK cells exhibited significant killing of A549 cells (FIG. 3D). In addition, the expression of cytolytic molecules, cytokines, and chemokines was analyzed after activating NK cells for 4 hours with A549 cells using the IsoLight single cell secretomics instrument. On average, HER2-CAR NK cells secreted granzyme B, perforin, TNF-α, MIP1-β, MCP-1, RANTES, and IL-8 at exceptionally increased levels; in contrast, less than 13% of unmodified NK cells secreted Granzyme B and less than 3.5% secreted the other cytokines. MIP1-α and IFN-γ were nearly identical between all groups. Additionally, some HER2-CAR NK cells could produce upwards of 10 effector molecules simultaneously. However, the majority produced 5 effector molecules or less (FIGS. 3E-3F). In contrast, unmodified NK cells were only capable of secreting on average 2 to 3 effector molecules at a far lower frequency. Activating iMC did not have a significant impact on effector molecule production in this 4-hour co-culture assay (FIGS. 3E-3F). To demonstrate the reproducibility that TBK1/IKKε inhibition enhances VSV-G LV transduction at various MOIs (10-30), obtained transduction efficiencies were compiled from studies disclosed herein (FIG. 3G). The mean transduction efficiency was 34.22% (range: 18-53.5%), which was significantly greater (p<0.0001) than for NK cells that were transduced without TBK1/IKKε inhibition (mean: 4.39%, range: 1.12-7.19%).

### 3. Discussion

The studies described herein above exemplify the disclosed method for NK cell transduction with VSV-G LVs that utilizes a transient antiviral pathway blockade. The studies demonstrate that that blocking TBK1/IKKε is essential for the observed benefit. In the studies, three compounds were assessed: (a) BX795, which is a broad-spectrum inhibitor of PDK1, ULK1, and NF-kB activation; (b) MRT67307, which is a relatively specific inhibitor of TBK1/IKKε; and (c) amlexlanox (Aphthasol™), an FDA approved drug that is a TBK1/IKKε inhibitor (Ref. 32). The data herein show that LentiBOOST™ did not further enhance transduction efficiency, suggesting that viral binding to the cell surface is not limiting NK cell transduction by VSV-G LVs. In addition, based on the data disclosed herein, it is possible that other more ‘innate-like’ cell populations may benefit from antiviral pathway inhibition during VSV-G LV transduction including γδ T cells (Refs. 34-35).

The disclosed method comprising TBK1/IKKε inhibition, as demonstrated in the examples above, enables the transduction of NK cells with VSV-G LVs. Moreover, the disclosed method could readily be translated into clinical grade production of genetically modified NK cells, and thus has the potential to have a significant impact on the rapidly expanding NK cell therapy field.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method for transducing cells comprising:
contacting cells with a transduction composition and a TBK1/IKKε inhibitor; and
wherein the TBK1/IKKε inhibitor comprises a substituted N-(3-((2-((3-(aminomethyl)-phenyl)amino)-5-methylpyrimidin-4-yl)amino)propyl)acetamide analogue;
thereby transducing the cells.

2. The method of claim 1, wherein the transduction composition is a virus particle.

3. The method of claim 2, wherein the virus particle comprises a lentiviral vector.

4. The method of claim 3, wherein the lentiviral vector comprises at least one transgene.

5. The method of claim 4, wherein the transgene is from about 100 base pairs to about 5,000 base pairs.

6. The method of claim 2, wherein the virus particle is a VSV-G pseudotyped LV particle.

7. The method of claim 2, wherein about 10% to about 100% of the cells contacting the virus particle and the TBK1/IKKε inhibitor are transduced with the virus particle.

8. The method of claim 7, wherein about 30% to about 100% of the cells contacting the virus particle and the TBK1/IKKε inhibitor are transduced with the virus particle.

9. The method of claim 7, wherein at least about 30% of the cells contacting the virus particle and the TBK1/IKKε inhibitor are transduced with the virus particle.

10. The method of claim 7, wherein at least about 40% of the cells contacting the virus particle and the TBK1/IKKε inhibitor are transduced with the virus particle.

11. The method of claim 7, wherein at least about 50% of the cells contacting the virus particle and the TBK1/IKKε inhibitor are transduced with the virus particle.

12. The method of claim 2, wherein the contacting the cells with a virus particle is at a multiplicity of infection (MOI) of about 1 to about 100.

13. The method of claim 12, wherein the MOI is about 1 to about 10.

14. The method of claim 12, wherein the MOI is about 2 to about 7.

15. The method of claim 12, wherein the MOI is about 1 to about 5.

16. The method of claim 1, wherein the transduction composition is a ribonucleoprotein (RNP) complex.

17. The method of claim 16, wherein the ribonucleoprotein (RNP) complex is a CRISPR ribonucleoprotein (RNP) complex.

18. The method of claim 1, wherein the transduction composition is a protein.

19. The method of claim 18, wherein the protein is an antibody.

20. The method of claim 1, wherein the transduction composition is selected from a plasmid, DNA fragment, oligonucleotide, RNA, and combinations thereof.

21. The method of claim 1, wherein the substituted N-(3-((2-((3-(aminomethyl)phenyl)amino)-5-methylpyrimidin-4-yl)amino)propyl)acetamide analogue has a structure given by the formula: 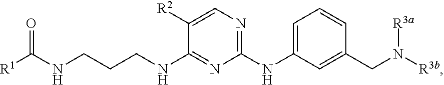
wherein R¹ is an alkyl, heteroalkyl, cycloalkyl, cycloheteroalkyl, aryl, heteroaryl, or substituted forms of any of the foregoing; wherein R² is an alkyl, heteroalkyl, cycloalkyl, cycloheteroalkyl, aryl, heteroaryl, or substituted forms of any of the foregoing; and wherein each of R^{3a} and R^{3b} are independently selected from hydrogen, alkyl, heteroalkyl, cycloalkyl, cycloheteroalkyl, aryl, heteroaryl, or substituted forms of any of the foregoing.

22. The method of claim 21, wherein the substituted N-(3-((2-((3-(aminomethyl)-phenyl)amino)-5-methylpyrimidin-4-yl)amino)propyl)acetamide analogue has a structure given by the formula:

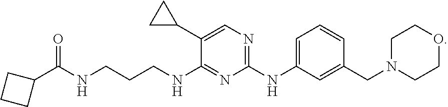 [SMILES: O=C(NCCCNc1nc(Nc2cccc(CN3CCOCC3)c2)ncc1C1CC1)C1CCC1]

23. The method of claim 1, wherein the method does not utilize a toll-like receptor inhibitor.

24. The method of claim 1, wherein the method does not utilize a cationic additive.

25. The method of claim 1, wherein the cells are NK cells.

26. The method of claim 1, wherein the cells are innate cells.

27. The method of claim 26, wherein the innate cells are γδ T cells.

28. The method of claim 1, wherein the transducing comprises electroporation.

29. A kit comprising:
a TBK1/IKKε inhibitor; and
instructions for transducing NK cells;
wherein the TBK1/IKKε inhibitor comprises a substituted N-(3-((2-((3-(aminomethyl)-phenyl)amino)-5-methylpyrimidin-4-yl)amino)propyl)acetamide analogue.

30. The kit of claim 29, further comprising a virus particle comprising a lentiviral vector.

31. The kit of claim 29, further comprising NK cells.

32. The kit of claim 29, wherein the instructions comprise the method of claim 1.
